(19) United States
(12) Patent Application Publication
(10) Pub. No.: US 2018/0263792 A1
(43) Pub. Date: Sep. 20, 2018

(54) ARTIFICIAL FOOT

(71) Applicant: C LindheXtend AB, Halmstad (SE)

(72) Inventors: Christoffer LINDHE, Halmstad (SE); Anders KLINTANG, Alvsjo (SE)

(21) Appl. No.: 15/981,149

(22) Filed: May 16, 2018

Related U.S. Application Data
(63) Continuation of application No. 14/778,204, filed on Sep. 18, 2015, now abandoned.

(30) Foreign Application Priority Data
Mar. 18, 2013 (SE) 1350323-0

(51) Int. Cl. A61F 2/66; A61F 2/50

(52) U.S. Cl. CPC A61F 2/66; A61F 2002/6657; A61F 2002/6642; A61F 2002/6621; A61F 2002/5007

(57) ABSTRACT

A prosthetic foot device having a toe region, an ankle region and a heel region, said device comprising
an attachment unit configured to be connected to a coupling device and/or to stump of an amputee;
a resilient upper forefoot member extending from the attachment unit through the ankle region towards the toe region, said forefoot member comprising at least one elongated curvilinear forefoot leaf spring and at least one longitudinally extending parting slot dividing the toe region in at least two parts:
a resilient heel member extending from the attachment unit through the ankle region towards the heel region, said heel member comprising at least one elongated curvilinear heel leaf spring;
a resilient lower foot member attached to the forefoot member at said toe region and to said heel member in the heel region, wherein said lower foot member is laterally unitary along the longitudinal length thereof.

This application is a continuation of U.S. application Ser. No. 14/778,204 filed Sep. 18, 2015 which is a national phase of International Application No. PCT/EP2014/055411 filed Mar. 18, 2014 and published in the English language, and claims priority to Swedish Application No. 1350323-0 filed Mar. 18, 2013, which are hereby incorporated by reference in their entirety.

### TECHNICAL FIELD

The invention relates generally to lower leg prostheses and, more particularly, to a lower prosthetic foot that simulates the performance characteristics of a natural foot.

### BACKGROUND OF THE INVENTION

A prosthetic foot is a very important constituent of a leg prosthesis. A prosthetic foot must reliably store and release energy while flexing in a number of degrees of motion so as to properly coordinate with the muscular action when the user is walking, running or standing. In addition it is desirable that the prosthetic foot is relatively inexpensive. Significant advancements have been made in the field during recent years, many of which are mechanically complex and employ a number of moving parts. While such devices provide good and reliable performance characteristics, their cost and complexity limit their use particular in high volume applications and in user communities, which do not have a sophisticated technical infrastructure to support and maintain such devices.

The development of composite materials technology, such as fiberglass/epoxy and carbon fiber/epoxy composite materials and their incorporation into prosthetic feet have improved the performance characteristics and feel of a prosthetic foot, resulting in an improved mobility for the wearer. However, few designs have focused on the lateral, or side to side rotation when the foot is used on varied or uneven terrain. The forefoot and heel of a natural foot rotates with a medial to lateral roll-over to accommodate variations in terrain. Many artificial feet of previous designs usually incorporate a unitary foot that is incapable of such lateral roll-over movement.

The object of the present invention is to provide a prosthetic foot which does not include any articulate spring members but which emulates containing articulate spring members, while still providing a comfortable and natural foot action. The prosthetic foot of the invention does not require any periodic maintenance or adjustment, and has relatively low production costs. Furthermore, due to the specific design of the prosthetic foot it offers an improved roll-over movement and stability when used on uneven terrain.

### SUMMARY OF THE INVENTION

This object may be achieved by a prosthetic foot device having a toe region, an ankle region and a heel region, corresponding in location to a toe region, an ankle region, and a heel region respectively of a natural foot. The prosthetic foot device has a longitudinal extension from the heel region to the toe region corresponding to the longitudinal extension of a natural foot, and a lateral extension extending across the prosthetic device which is perpendicular to the longitudinal direction and corresponding to the width of a natural foot. The prosthetic foot device comprises an attachment unit configured to be connected to a coupling device, and/or to a stump of an amputee. The attachment unit is advantageously located in the ankle region or at a location above the ankle of a natural foot.

The prosthetic foot device further comprises a resilient upper forefoot member extending forwardly and downwardly from the attachment unit through the ankle region towards the toe region, said forefoot member comprising at least one elongated curvilinear forefoot leaf spring and at least one longitudinally extending parting slot dividing the toe region in at least two parts.

The device has a resilient heel member extending forwardly and downwardly from the attachment unit towards the toe region, curving through the ankle region and thereafter extending rearward towards the heel region, said heel member comprising at least one elongated curvilinear heel leaf spring. The prosthetic foot device also has a resilient lower foot member with a lower surface configured to engage the ground or surface for walking. The lower foot member is disposed below the forefoot and heel members of the prosthetic foot device. Generally the forefoot and heel members of the prosthetic foot device are located in the vertical direction above the lower foot plate when the lower surface of the lower foot plate engages a substantially horizontal ground in walking. Alternatively the lower surface of the lower foot member may engage the inside of a cosmesis or a shoe. The upper surface of the resilient lower foot member is attached to the forefoot member at said toe region and to the heel member in the heel region, and is laterally unitary along the longitudinal length thereof.

The resilient upper forefoot member is connected to, and extends from the attachment unit through the ankle region towards the toe region, said forefoot member comprising at least one elongated curvilinear forefoot leaf spring. The at least one curvilinear forefoot leaf spring is coupled to the attachment unit and terminates at a toe location corresponding to that of a natural foot. The attachment unit has an upper end configured to be connected to a coupling device, and/or to a stump of an amputee, and a bottom surface facing the upper forefoot member. Advantageously the bottom surface of the attachment unit is chamfered to coincide with the sloping angle of the upper forefoot member as this extends forwardly and downwardly from the attachment unit through the ankle region and towards the toe region. The upper end of the attachment unit is advantageously modifiable to adjust the angle between the prosthetic foot device and the coupling device, and/or to a stump of an amputee.

The prosthetic foot device contains a resilient heel member which extends from the attachment unit through the ankle region towards the heel region, said heel member comprising at least one elongated curvilinear heel leaf spring. Said at least one elongated curvilinear heel leaf spring may be connected to the forefoot member or coupled directly to the attachment unit. It extends downward and forwardly towards the toe region, curves around the ankle region and extends rearward towards the heel region located at a heel location of a natural foot.

The prosthetic foot device further contains a resilient lower foot member extending from a toe region to a heel region, located at a toe and heel location respectively of a natural foot. The resilient lower foot member is attached to the forefoot member at said toe region, and to the heel member at the heel region. The lower foot member is laterally unitary along the longitudinal length thereof, i.e. it contains no parting slots. The resilient lower foot member extends a longitudinal length corresponding to that of a natural foot, and can be essentially flat and horizontal. However, the lower foot member may have curvatures including a downward curvature or valley in the area of a ball and heel location, and upward curvatures or peaks at the toe and arch locations of a natural foot.

The forefoot and heel leaf springs, as well as the lower foot member mentioned above, are made from a material that permit them to bend and deflect under force to store energy, and thereafter return to their original shape releasing energy as the force is removed. The leaf springs are advantageously flexible and resilient, and can be a composite, and include fibers, such as graphite fibers in a resin matrix. The springs can be straight or flat in the lateral, or side to side direction, while being curved in the longitudinal, or toe to heel direction.

The at least one leaf spring of the upper forefoot member has at least one longitudinally extending parting slot dividing the toe region in at least two parts. Advantageously, the at least one forefoot member contains one longitudinally extending parting slot, dividing the toe region into two parts. Thus, the at least one forefoot leaf spring of the forefoot member is vertically bifurcated (i.e. It has a cut through the leaf spring) extending from the toe end towards the attachment unit. The parting slot extends along the longitudinal length of the forefoot member, dividing the toe region in substantially two or more equal parts in the lateral direction. In one embodiment of the invention the parting slot may be located a bit off-center, i.e. the parting slot divides the toe region into two unequal parts in the lateral direction. Advantageously the part corresponding to the inside part of a natural foot is smaller, thereby simulating a big toe of a natural foot. In one embodiment the at least one forefoot member contains two longitudinally extending parting slots, dividing the toe region into three parts. In one further embodiment the at least one forefoot member contains three longitudinally extending parting slots, dividing the toe region into four parts.

The one or more parting slots allow the toe region to flex more independently, more like a natural foot and permit the foot to respond to uneven terrain. However, it is important that the at least one leaf spring having the at least one parting slot is connected to a lower foot member which is unitary in the toe region, i.e. it contains no parting slots. The advantages of having a unitary lower foot member (i.e. a leaf spring without any parting slot), compared to one comprising parting slots are many. With a unitary lower foot member as the one in the foot prosthetic device of the invention, the foot prosthesis device is endowed with a resilience which is more evenly distributed across the entire forefoot during load when the prosthesis is biased.

Furthermore, with a unitary lower foot member, the foot prosthetic device becomes more stable without having to increase the thickness of any of the leaf springs. If both the forefoot leaf spring and the lower foot member are bifurcated, the leaf springs must be thicker to provide the required stability to the foot prosthesis. An increased thickness of the leaf springs leads to a foot prosthesis with an overall increased stiffness and less resiliency. This was proven in a trial, wherein a person of 60 kg, when walking on a smooth terrain, stepped on a small obstacle 1.5 cm high. When wearing a prosthetic foot wherein both the forefoot member and the lower foot member contained a parting slot, the prosthetic foot adjusted 7° when the wearer stepped on the obstacle. This should be compared to an adjustment of 14-15°, when wearing the prosthetic foot of the invention.

The lower foot member is connected to the forefoot member and the heel member by elastomeric adhesive having a Young's modulus (E) of less than 13 MPa. As used herein the term Young's modulus (also known as tensile modulus) is intended to mean the measure of the stiffness of an elastic material, such as the elastomeric adhesive that is used to connect the lower foot member to the forefoot and heel members. The Young's modulus calculates the change in the dimension of a piece made of an isotropic elastic material under tensile or compressive loads. For instance, it predicts how much a material sample, e.g. the adhesive, extends under tension or shortens under compression. The Young′ modulus is expressed in units of pressure, pascal (Pa or N/m² or m⁻¹×kg×s⁻²), but the more practical unit when used in this invention is megapascal (Mpa or N/mm²).

A stiff material has a high Young's modulus and changes its shape only slightly under elastic loads (e.g. diamond). A flexible material has a low Young's modulus and changes its shape considerably (e.g. rubber). The elastomeric adhesive used to connect the lower foot member to the forefoot and heel members should be flexible enough to provide the foot with resiliency and flexibility, yet stiff enough to withstand the load of a wearer during regular use, such as standing, walking, running and jumping. Advantageously, the elastomeric adhesive used to connect the lower foot member to the forefoot and heel members should have a Young's modulus (E) of less than 13 MPa.

The at least one parting slot has a length that may extend from an anterior toe region along 15-50%, more preferably 25-35%, most preferably about 30% of the length of the forefoot member. This means that less than half and more preferably around one third of the forefoot member in the longitudinal direction contains the one or more parting slots.

The elastomeric adhesive is applied to first bonding areas between the forefoot member and the lower foot member, said first bonding areas extending in the longitudinal direction from the anterior toe region along 25-60%, more preferably along 30-55%, most preferably 40-50% of the length of the at least one parting slot of the forefoot member and towards the ankle region, and 30-60%, preferably 40-55%, and most preferably 50% of the width in the lateral directions of said foot prosthesis from said parting slot.

This means that the elastomeric adhesive is applied in only part of a possible first contact area, i.e. the area available for contact between the forefoot member and the lower foot member. Said first contact area covers the area between the forefoot member and the lower foot member, extending from side to side in the lateral direction and from the anterior toe region and in the longitudinal direction of the foot until the forefoot member curves and deflects upwards away from the lower foot member. Said first bonding areas between said forefoot and lower foot members extend from the anterior toe region, i.e. the foremost end of the toe region and in the longitudinal direction of the forefoot member towards the ankle region along 25-60%, more preferably along 30-55%, most preferably 40-50% of the length on each side of the at least one parting slot, and in the lateral direction starting from both sides of the one or more parting slots covering 30-60%, preferably 40-55%, and most preferably 50% of the width in said lateral directions. This means that the first bonding areas generally cover the areas extending from the anterior toe region and close to each side of the parting slot. The outermost areas between the lower foot member and the forefoot members in the lateral directions from the parting slot are free from elastomeric adhesive, allowing the forefoot member and lower foot member to move freely with respect to each other in the toe region. However, it is important that no elastomeric adhesive is applied to the parting slot itself.

The elastomeric adhesive is applied to a second bonding area between the forefoot member and the lower foot member in the heel region.

The forefoot member may comprise at least two elongated curvilinear forefoot leaf springs connected in the toe region, forming furcated forwardly slightly concave arcs, that may define gap between the at least two forefoot leaf springs in the ankle region. Advantageously, the forefoot member comprises two forefoot leaf springs forming vertically bifurcated forwardly concave arcs which define a gap between said two forefoot leaf springs in the ankle region. The gap between the at least two forefoot leaf springs endows said forefoot member with an improved resiliency, as the forefoot leaf springs, independently from each other, deflect under force to store energy, and return to their original shape releasing energy as the force is removed, thereby improving the overall resiliency of the foot prosthesis. Advantageously there is a damper located between each of the at least two forefoot leaf springs at the location where the forefoot leaf springs are attached to the attachment unit. The dampers will aid in providing a gap between said forefoot leaf springs.

The forefoot leaf springs of the forefoot member are connected by elastomeric adhesive applied to third bonding areas, said third bonding areas extending in the longitudinal direction from the anterior toe region along 25-60%, more preferably along 35-55%, most preferably 40-50% of the length of the parting slots of the forefoot leaf springs and towards the ankle region, and at least 50%, preferably at least 75%, and most preferably at least 90% of the width in the lateral directions from said parting slots on the forefoot leaf springs.

This means that the elastomeric adhesive is applied in only part of a possible third contact area. Said third contact area covers the area available for contact between the forefoot leaf springs, i.e. the entire toe region between the forefoot leaf springs, extending from one side to the other side of the forefoot leaf springs in the lateral direction, and from the anterior toe region in the longitudinal direction of the foot until the forefoot leaf springs curve and deflect away from teach other in the ankle region. Said third bonding areas between the forefoot leaf springs extend from the anterior toe region, i.e. the foremost end of the toe region and in the longitudinal direction of the forefoot leaf springs towards the ankle region, along 25-60%, more preferably along 35-55%, most preferably 40-50% of the length on each side of the at least one parting slot, and in the lateral direction starting from both sides of the one or more the parting slots, covering at least 50%, preferably at least 75%, and most preferably at least 90% of the width in said lateral directions. This means that the third bonding area generally covers the area extending from the anterior toe region and adjacent to both sides of the parting slot. The outermost areas in the lateral direction between the two forefoot leaf springs are free from elastomeric adhesive, allowing the forefoot leaf springs to move freely with respect to each other along the outer edges in the toe region. However, it is important that no elastomeric adhesive is applied to the parting slot itself.

The heel member may comprise at least two elongated curvilinear heel leaf springs which extend from the attachment unit downwardly and forwardly towards the toe region, curve around the ankle region and thereafter extend rearward towards the heel region. Advantageously the heel member comprises two elongated curvilinear heel leaf springs connected in the heel region defining a gap between said heel leaf springs in the ankle region. The gap between the at least two forefoot leaf springs endows said heel member with an improved resiliency as the leaf springs, independently from one another, deflect under force to store energy, and return to their original shape releasing energy as the force is removed. Said heel leaf springs are connected to each other by elastomeric adhesive applied to a fourth bonding area.

The elastomeric adhesive applied to the first and second bonding areas may be a first elastomeric adhesive having a lower tensile modulus (E) than a second elastomeric adhesive applied to the third and fourth bonding areas.

Advantageously, elastomeric adhesives having different tensile moduli are used in the different bonding areas to improve the resiliency and flexibility of the prosthetic foot during gait.

Using an elastomeric adhesive with higher tensile modulus will improve the stiffness and also the durability of the bonding regions, but will at the same time make the bond less resilient. An elastomeric adhesive with a low tensile modulus will improve the resiliency considerably, but decrease the durability of the bonding region. Thus, the use of elastomeric adhesives in the present invention is a delicate balance between providing a prosthetic foot which has maximum resiliency for optimal gait, yet which is stiff enough to provide the durability required for a foot which is constantly exposed to strain. Advantageously the first elastomeric adhesive has a tensile modulus (E) of less than 5 MPa, more preferably, less than 3 MPa, most preferably about 1 MPa, and advantageously the second elastomeric adhesive has a tensile modulus (E) of 13-7 MPa, more preferably, 12-8 MPa, most preferably about 10 MPa.

The forefoot leaf springs, heel leaf springs and the lower foot member are made from a flexible and resilient material, such as a composite material with fibres in a resin matrix. Advantageously the flexible and resilient material is carbon fibres or fiberglass.

The specific design of the prosthetic foot device allows the at least two forefoot spring leafs of the forefoot member and the lower foot member to cooperate during gait and thereby create a stable, yet at the same time a flexible prosthesis foot with an improved adaptability to the terrain. When a prosthesis foot made from less flexible or thicker spring leafs are biased due to an uneven ground or a pebble, it is too stiff to adapt to the obstacle, and a levering action will transplant itself through the body trying to reposition the body sidewise in order for the foot to establish contact with the ground. The levering action will make the user try to cancel the levering force, which has the result that he will end up standing on the top of the obstacle instead.

Thin spring leafs, together with the bifurcated toe region of the forefoot member being attached to a unitary lower foot member as well as the use of elastomeric adhesives with different tensile modulus attaching said lower foot member to the forefoot and heel members, will work together and form a prosthetic foot that offers an improved roll-over movement and stability when used on uneven terrain.

### BRIEF DESCRIPTION OF THE DRAWINGS

FIG. 1 is a perspective view of an embodiment of the prosthetic foot device of the invention

FIG. 2 is a side view of the prosthetic foot device of FIG. 1

FIG. 3 is a bottom view of the prosthetic foot device of FIG. 1

FIG. 4 is an exploded view of the prosthetic foot device of FIG. 1

FIG. 5 is a perspective view of a further embodiment of the prosthetic foot device of the invention.

FIG. 6 is a top view of the prosthetic device of FIG. 5.

### DETAILED DESCRIPTION OF PREFERRED EMBODIMENTS

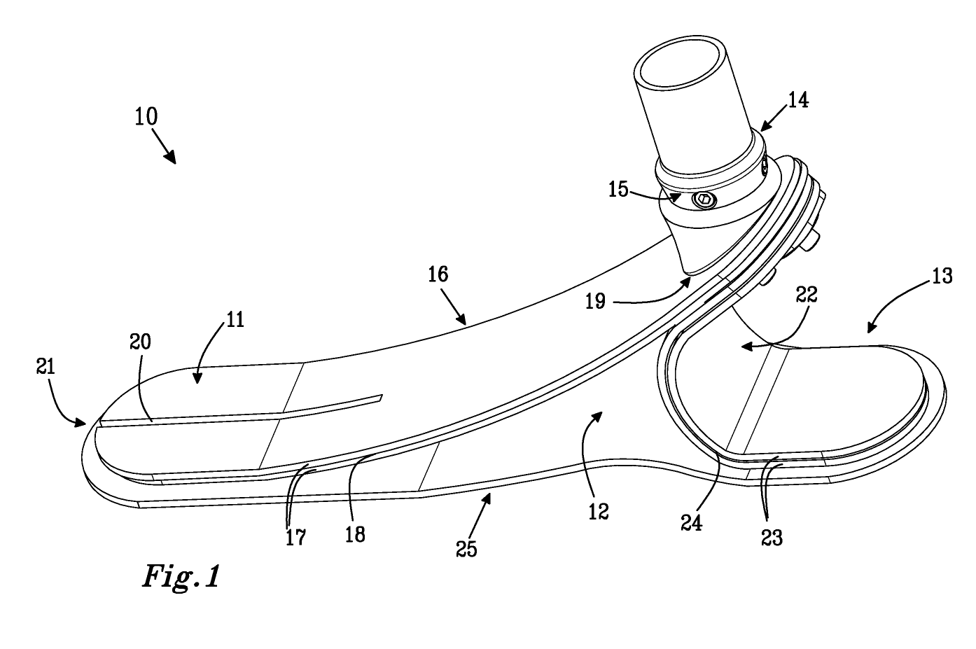

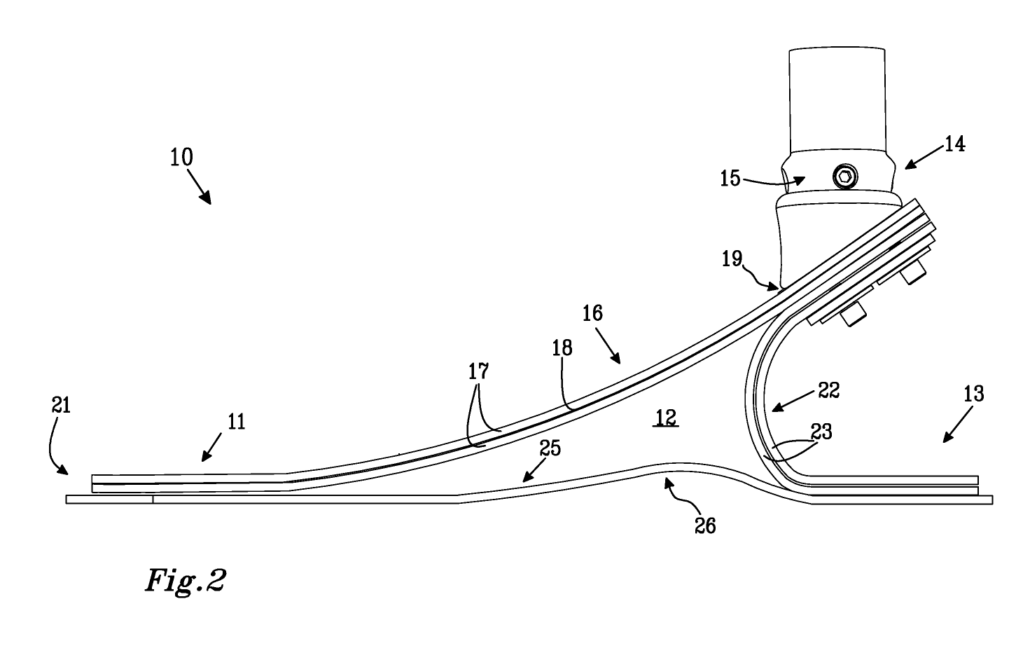

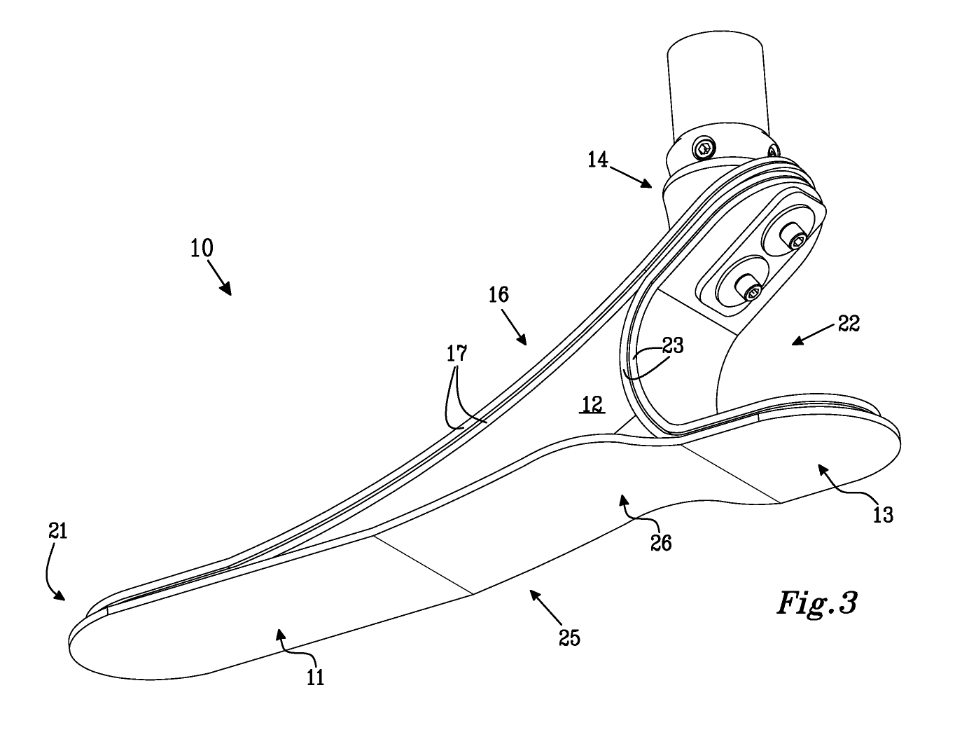

In the following the invention will be described in a non-limiting way and in more detail with reference to exemplary embodiments illustrated in the enclosed drawings 1-4, in which FIGS. 1-3 show a schematic view of the prosthesis foot 10 of the invention, as seen from different angles. The prosthetic foot device 10 of the present invention has a toe region 11, an ankle region 12 and a heel region 13, corresponding to a toe location, an ankle location, and a heel location respectively of a natural foot. The prosthetic foot device 10 comprises an attachment unit 14 configured to be connected to coupling device (not shown) and/or to a stump of an amputee. The upper end of the attachment unit 15 may be adjustable to modify the angle between the prosthetic foot 10 and the leg of the amputee (not shown).

A resilient upper forefoot member 16 comprising at least one elongated curvilinear forefoot leaf spring 17 is connected to, and extends from the attachment unit 14 through the ankle region 12 towards the toe region 11. Advantageously, the forefoot member 16 comprises at least two elongated curvilinear forefoot leaf springs 17 connected in the toe region 11, forming two slightly concave arcs, defining a first gap 18 between the two forefoot leaf springs 17 in the ankle region 12.

In this embodiment the two curvilinear forefoot leaf springs 17 are coupled to the attachment unit 14, extend forwardly and downwardly and terminate at the region of a toe location of a natural foot. The bottom surface of the attachment unit 19 is chamfered to coincide with the same sloping angle as the upper forefoot member 16. A damper (not shown) may be arranged between the two forefoot leaf springs 17 at the attachment unit 14 to provide a gap 18 between said forefoot leaf springs 17, as well as preventing any rubbing of said leaf springs against each other, at the connection with the attachment unit 14.

The least one forefoot leaf spring 17 of the upper forefoot member 16 has at least one longitudinally extending parting slot 20 dividing the toe region 11 in at least two parts. Advantageously the upper forefoot member 16 has one parting slot 20 as seen in FIGS. 1-4. The parting slot 20 has a length that extends from the anterior toe region 21 along 15-40%, more preferably 25-35%, most preferably about 30% of the length of the forefoot member 16. This means that less than half and more preferably around one third of the forefoot member 16 in the longitudinal direction contains a parting slot 20.

The prosthetic foot device 10 contains a resilient heel member 22 comprising at least one elongated curvilinear heel leaf spring 23, connected to and extending from the attachment unit 14 through the ankle region 12 towards the heel region. FIGS. 1-4 disclose a heel member 22 of a prosthetic foot with two elongated curvilinear heel leaf springs 23 which extend from the attachment unit 14 downward and forwardly towards the toe region, curve around the ankle region 12 and rearward towards the heel region 13. A second gap 24 is formed between the two heel leaf springs 23 in the ankle region 12. A damper (not shown) may be arranged between the two heel leaf springs 23 at the attachment unit 14 to provide a gap between said heel leaf springs 23, as well as preventing any rubbing of said leaf springs against each other, at the connection with the attachment unit 14.

The prosthetic foot device 10 further contains a resilient and laterally unitary lower foot member 25 extending from a toe region 11 to a heel region 13, located at a toe and heel location respectively of a natural foot. The resilient lower foot member 25 is attached to the forefoot member 16 in said toe region 11, and to the heel member 22 in the heel region 13. The resilient lower foot member 25 extends a longitudinal length of a foot, and is essentially flat and horizontal, but may have curvatures including a downward curvature or valley 26 in the area of a ball and heel location, and upward curvatures or peaks 27 at the toe and arch locations of a natural foot.

The lower foot member 25 is connected to the forefoot member 16 and the heel member 22 by means of elastomeric adhesive having a Young's modulus (E) of less than 13 MPa.

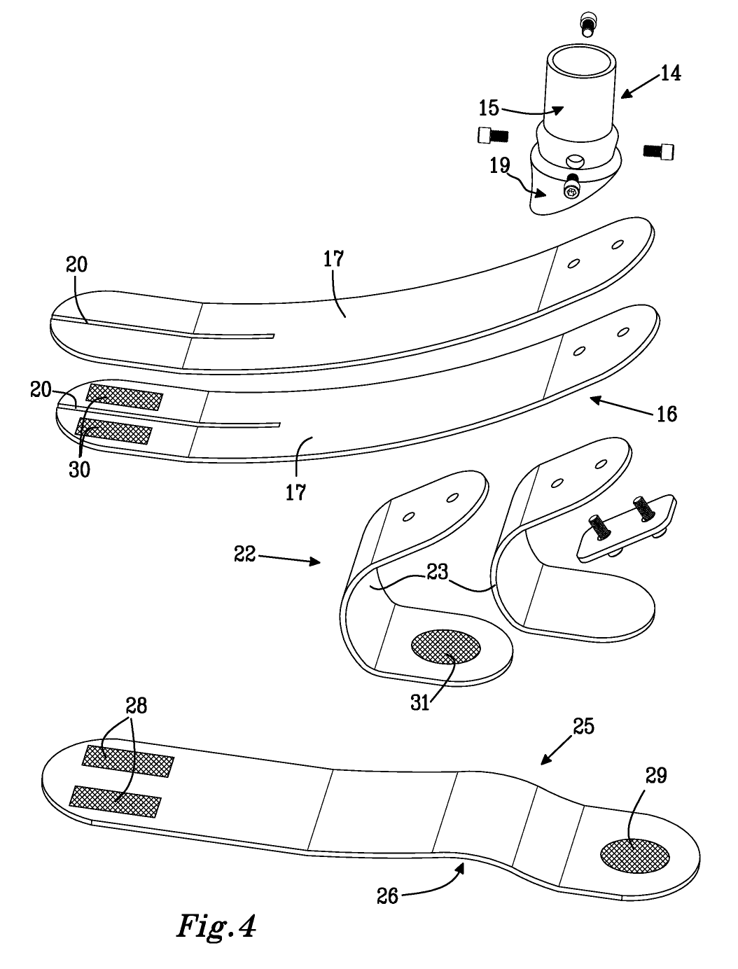

The elastomeric adhesive is applied to first bonding areas 28 between the forefoot member 16 and the lower foot member 25. As can be seen in FIG. 4, said first bonding areas 28 extend in the longitudinal direction from the anterior toe region 20 along 25-60%, more preferably along 35-55%, most preferably 40-50% of the length of the at least one parting slot 20 of the forefoot member 16 and towards the ankle region 12, and 30-60%, preferably 40-55%, and most preferably 50% of the width in the lateral directions of said foot prosthesis from said parting slot 20. The outermost areas between the lower foot member 25 and the forefoot member 16 in the lateral directions from the parting slot 20 are free from elastomeric adhesive, allowing the forefoot member 16 and lower foot member 25 to move freely with respect to each other in the toe region 11. However, it is important that no elastomeric adhesive is applied to the parting slot 20 itself. The elastomeric adhesive is applied to a second bonding area 29 between the forefoot member 16 and the lower foot member in the heel region 13.

The forefoot leaf springs 17 of the forefoot member 16 are connected by elastomeric adhesive applied to third bonding areas 30. As can be seen in FIG. 4, said third bonding areas 30 extend in the longitudinal direction from the anterior toe region 20 along 25-60%, more preferably along 35-55%, most preferably 40-50% of the length of the parting slots 20 of the forefoot leaf springs 17 and towards the ankle region 12, and at least 50%, preferably at least 75%, and most preferably at least 90% of the width in the lateral directions from said parting slots 20 on the forefoot leaf springs 17. The heel leaf springs 23 are connected to each other by elastomeric adhesive applied to a fourth bonding area 31.

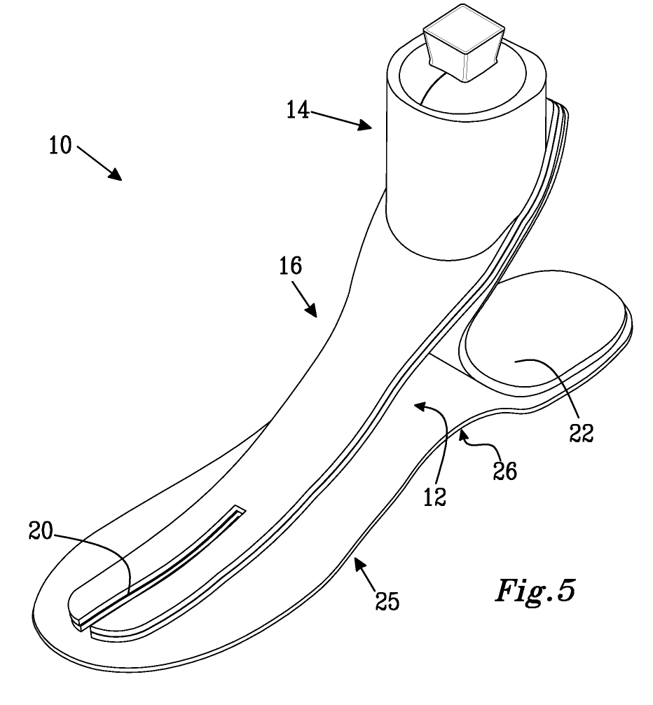

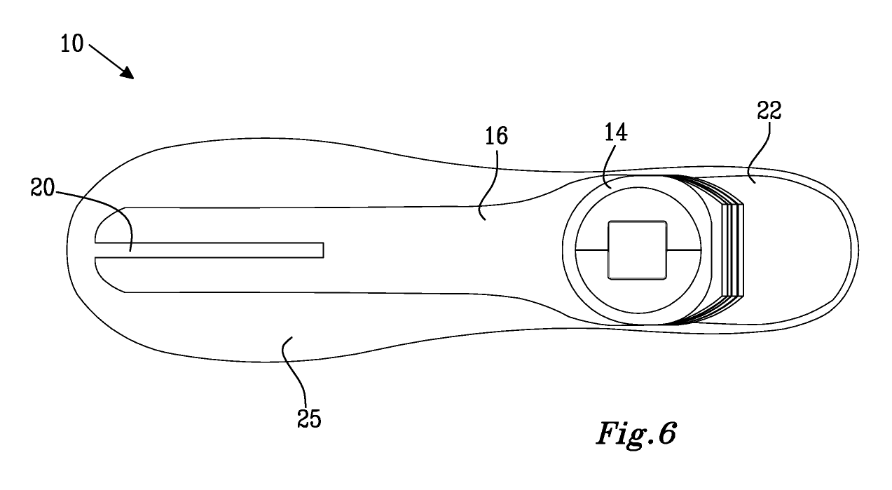

FIGS. 5 and 6 disclose a further embodiment of the prosthetic foot device 10. In this embodiment the forefoot member 16 and the heel member 22 are narrower than the lower foot member 25. The narrower shapes of the forefoot member 16 and in particular the heel member 22 provide the prosthetic device with an enhanced ability to bend or twist sideways during gait. Furthermore, the lower foot member 25 is provided with a “waist”, i.e. a narrowing of the foot member 25 in the lateral direction, located in between the toe region and the heel region of the lower foot member 25. This “waist” enables the prosthetic foot device to better adjust to any unevenness on the ground.

1. A prosthetic foot device having a toe region, an ankle region and a heel region, said device comprising
an attachment unit configured to be connected to a coupling device and/or to a stump of an amputee;
an upper forefoot member extending from the attachment unit through the ankle region towards the toe region, said forefoot member comprising at least one elongated curvilinear forefoot leaf spring and at least one longitudinally extending parting slot dividing the toe region of the fore foot member in at least two parts, the slot extending through the entire thickness of the forefoot leaf spring;
a heel member extending from the attachment unit through the ankle region towards the heel region, said heel member comprising at least one elongated curvilinear heel leaf spring;
a non-furcated lower foot member attached to the forefoot member at said toe region and to said heel member in the heel region.

2. The prosthetic foot device according to claim 1, wherein the at least one parting slot has a length extending from an anterior toe region towards the attachment unit along 15-50% of the longitudinal length of the forefoot member.

3. The prosthetic foot device according to claim 1, wherein the lower foot member is connected to the forefoot member and the heel member by an elastomeric adhesive having a tensile modulus (E) of less than 13 MPa.

4. The prosthetic foot device according to claim 3, wherein the elastomeric adhesive is applied to first bonding areas between the forefoot member and the lower foot member in the toe region, said first bonding areas extending in the longitudinal direction from the anterior toe region along 25-60%-of the length of the at least one parting slot of the forefoot member and towards the ankle region, and 30-60% of the width in the lateral directions from said parting slot.

5. The prosthetic foot device according to claim 4, wherein the elastomeric adhesive is applied to a second bonding area between the forefoot member and the lower foot member in the heel region.

6. The prosthetic foot device according to claim 5, wherein the forefoot leaf springs of the upper forefoot member are connected by elastomeric adhesive applied to third bonding areas, said third bonding areas extending in the longitudinal direction from the anterior toe region along 25-60%-of the length of the at least one parting slot of the forefoot leaf springs and towards the ankle region, and at least 50% of the width from said parting slot.

7. The prosthetic foot device according to claim 1, wherein the heel member comprises at least two elongated curvilinear heel leaf springs extending from the attachment unit downward and forwardly towards the toe region, curving around the ankle region and rearward towards the heel region defining second gaps between the at least two heel leaf springs in the ankle region.

8. The prosthetic foot device according to claim 1, wherein the heel member comprises two elongated curvilinear heel leaf springs extending from the attachment unit downward and forwardly towards the toe region, curving around the ankle region and rearward towards the heel region defining a second gap between the two heel leaf springs in the ankle region.

9. The prosthetic foot device according to claim 6, wherein heel leaf springs are connected by elastomeric adhesive applied to a fourth bonding area.

10. The prosthetic foot device according to claim 9, wherein the elastomeric adhesive applied to the first and second bonding areas is a first elastomeric adhesive having a lower tensile modulus (E) than a second elastomeric adhesive applied to the third and fourth bonding areas.

11. The prosthetic foot device according to claim 10, wherein the first elastomeric adhesive has a tensile modulus (E) of less than 5 MPa.

12. The prosthetic foot device according to claim 10, wherein the second elastomeric adhesive has a tensile modulus (E) of 13-7 MPa.

13. The prosthetic foot device according to claim 1, wherein the forefoot leaf springs, heel leaf springs and the lower foot member are made from a flexible and resilient material.

14. The prosthetic foot device according to claim 13, wherein the flexible and resilient material is a composite material with carbon fibres in a resin matrix.

15. The prosthetic foot device according to claim 13, wherein the flexible and resilient material is carbon fibres or fiberglass.
